(19) United States
(12) Patent Application Publication
(10) Pub. No.: US 2016/0113888 A1
(43) Pub. Date: Apr. 28, 2016

(54) PHARMACEUTICAL COMPOSITION FOR TREATMENT OF CANCER

(71) Applicant: NATIONAL INSTITUTE OF ADVANCED INDUSTRIAL SCIENCE AND TECHNOLOGY, Chiyoda-ku, Tokyo (JP)

(72) Inventors: Renu WADHWA, Tsukuba-shi, Ibaraki (JP); Sunil KAUL, Tsukuba-shi, Ibaraki (JP)

(21) Appl. No.: 14/898,065

(22) PCT Filed: Jun. 11, 2014
(86) PCT No.: PCT/JP2014/066083

§ 371 Date: Dec. 11, 2015

(30) Foreign Application Priority Data
Jun. 14, 2013 (JP) 2013-125860

(51) Int. Cl. A61K 31/08

(52) U.S. Cl. CPC A61K 31/08

(57) ABSTRACT

An object of the present invention is to provide a novel drug for treating cancer. The present invention relates to a pharmaceutical composition for treating or preventing cancer which comprises, as an active ingredient, a triethylene glycol of the following formula (I) or a derivative thereof:

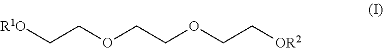

wherein R¹ and R² are independently selected from hydrogen, C₁₋₆ alkyl, C₁₋₆ haloalkyl, or —C(═O)R³; and R³ is selected from C₁₋₆ alkyl or C₁₋₆ haloalkyl.

### TECHNICAL FIELD

The present invention relates to a novel pharmaceutical composition for treatment of cancer.

### BACKGROUND ART

Cancer is a leading cause of death and accounts for a high percentage of death throughout the world. Although the survival rate of cancer patients has been increasing along with the progress in cancer diagnostic and treatment methods, it is still difficult for those living in developing countries, accounting for about 75% of the world population, to receive advanced cancer treatment. Therefore, inexpensive cancer therapies are in demand.

In contrast to advanced medical services, natural therapies with the use of herbs and the like have been known from old times. Ayurveda is a type of traditional Indian natural treatment dating back to before the Common Era. The roots of Ashwagandha (scientific name: Withania somnifera; also referred to as “Indian ginseng” or “Winter cherry”) used as a medical herb in Ayurveda are known to have nutritional fortification effects, health enhancement effects, and the like.

The present inventors focused on Ashwagandha leaves, which can be more easily collected than roots, and studied pharmacological effects of leaf extract. As the result, the present inventors previously found that a water extract of Ashwagandha leaves has anticancer activity (WO 2009/110546).

### SUMMARY OF INVENTION

### Technical Problem

However, it was unknown which component contained in a water extract of Ashwagandha leaves has anticancer activity. If an active ingredient contained in a water extract of Ashwagandha leaves could be identified, it would make it possible to provide a novel drug for cancer therapy based on the finding. Thus, an object of the present invention is to identify an active ingredient in a water extract of Ashwagandha leaves so as to provide a novel drug for cancer therapy containing based on the finding.

### Means for Solving the Problem

As a result of intensive studies of a water extract of Ashwagandha leaves, the present inventors successfully identified the active ingredient having anticancer activity and confirmed that the active ingredient actually has anticancer activity such as cytotoxicity to cancer cells. The present invention is summarized as follows.

(1) A pharmaceutical composition for treating or preventing a cancer, which comprises, as an active ingredient, a triethylene glycol of formula (I) or a derivative thereof:

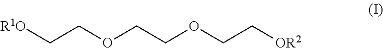

wherein
R¹ and R² are independently selected from hydrogen, C₁₋₆ alkyl, C₁₋₆ haloalkyl, or —C(═O)R³, and
R³ is selected from C₁₋₆ alkyl or C₁₋₆ haloalkyl.

(2) The pharmaceutical composition according to (1), wherein the cancer is a solid cancer.

(3) An agent for inhibiting cancer metastasis, which comprises, as an active ingredient, a triethylene glycol of formula (I) or a derivative thereof:

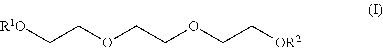

wherein
R¹ and R² are independently selected from hydrogen, C₁₋₆ alkyl, C₁₋₆ haloalkyl, or —C(═O)R³, and
R³ is selected from C₁₋₆ alkyl or C₁₋₆ haloalkyl.

(4) A method for treating or preventing cancer, which comprises administering to a subject in need thereof an effective amount of a triethylene glycol of formula (I) or a derivative thereof:

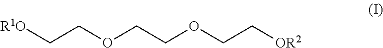

wherein
R¹ and R² are independently selected from hydrogen, C₁₋₆ alkyl, C₁₋₆ haloalkyl, or —C(═O)R³, and
R³ is selected from C₁₋₆ alkyl or C₁₋₆ haloalkyl.

### Effects of the Invention

According to the present invention, a novel drug for treating cancer can be provided.

This specification incorporates the content of the specification of Japanese Patent Application No. 2013-125860, for which priority is claimed to the present application.

### BRIEF DESCRIPTION OF THE DRAWINGS

FIG. 1 shows photo images demonstrating cytotoxicity assay results of AshLe-WEX (Water extract of Ashwagandha leaves) on human osteosarcoma cells (U2OS) and human breast cancer cells (MCF7). Control groups (C) shows human osteosarcoma cells (U2OS) and human breast cancer cells (MCF7) that were not treated with AshLe-WEX.

FIG. 2 shows photo images demonstrating cytotoxicity assay results of AshLe-WEX and AshLe-WEX whose protein components are inactivated on human osteosarcoma cells (U2OS) and normal human fibroblasts (TIG-1). AshLe-WEX used herein whose protein components are inactivated was obtained via heat denaturation (AshLe-WEX-HI) or proteinase degradation (AshLe-WEX-PI). Control groups are of human osteosarcoma cells (U2OS) and normal human fibroblasts (TIG-1) that were not treated with AshLe-WEX or AshLe-WEX whose protein components are inactivated.

FIG. 3 shows NMR charts of AshLe-WEX-F2 (the 2nd fraction from AshLe-WEX obtained by the fractional method described in Example 4 below) and triethylene glycol. In FIG. 3, charts (a) and (b) show analysis results of AshLe-WEX and charts (c) and (d) show analysis results of triethylene glycol.

FIG. 4 shows an HPLC chart of AshLe-WEX. Commercially available purified triethylene glycol was added as a standard substance.

FIG. 5 shows photo images of cytotoxicity assay results of triethylene glycol on human osteosarcoma cells (U2OS) and normal human fibroblasts (TIG-1).

FIG. 6 shows charts and a graph of cell cycle analysis results of human osteosarcoma cells (U2OS) that were treated with AshLe-WEX or triethylene glycol. A control group consists of human osteosarcoma cells (U2OS) that were not treated with AshLe-WEX or triethylene glycol.

FIG. 7 shows photo images and a graph of in vivo antitumor assay results of AshLe-WEX and triethylene glycol. In vivo antitumor assay was conducted by injecting HT1080 cells subcutaneously or via a tail vein into nude mice to cause tumorigenesis and then administering AshLe-WEX (oral administration) or triethylene glycol (oral administration (TEG) or intraperitoneal injection (TEG-ip)) to the nude mice. 2% carboxymethyl cellulose was orally administered instead of AshLe-WEX or triethylene glycol to mice of a control group after tumorigenesis. FIG. 7A shows photo images of the abdomens of mice after the elapse of the period of administration of 2% carboxymethyl cellulose (control group), administration of AshLe-WEX, oral administration of triethylene glycol (TEG), or intraperitoneal administration of triethylene glycol (TEG-ip). FIG. 7B shows a graph of results of time-dependent changes in the tumor volume determined during in vivo antitumor assay.

FIG. 8 shows results of in vivo tumor metastasis assay for determination of anti-cancer metastasis activity of triethylene glycol. FIG. 8A shows photos of lungs excised from mice after in vivo tumor metastasis assay (each circle indicates a tumor). The upper photo images show the lungs of the 2% carboxymethyl cellulose administration group (control group), the middle photo images show the lungs of the AshLe-WEX administration group, and the lower photo images show the lungs of the triethylene glycol administration group. FIG. 8B shows a graph of the mean lung tumor volumes for each group after in vivo tumor metastasis assay.

FIG. 9 shows a graph demonstrating the results of in vitro Matrigel invasion assay using HT1080 cells. Triethylene glycol (TEG) or AshLe-WEX was used as a test substance. Assay results for a control group of HT1080 cells that were not treated with triethylene glycol or AshLe-WEX are also shown.

FIG. 10A shows photo images and graphs of Western blotting determination results regarding the expression levels of cancer-inhibiting proteins (p53, p21, and pRB) in human osteosarcoma cells (U2OS) or normal human fibroblasts (TIG-1) that were treated with AshLe-WEX or triethylene glycol. FIG. 10B shows photo images and graphs of Western blotting determination results regarding the expression levels of cell cycle regulatory proteins (cyclin-B1, cyclin-D1, cyclin-E1, CDK-2, CDK-4, and CDK-6) in human osteosarcoma cells (U2OS) or normal human fibroblasts (TIG-1) that were treated with AshLe-WEX or triethylene glycol. FIGS. 10A and 10B also show the expression levels of the proteins in cells of a control group that were not treated with AshLe-WEX or triethylene glycol.

FIG. 11 shows photo images of results of immunohistochemistry conducted using an anti-pRB antibody on human osteosarcoma cells (U2OS) or normal human fibroblasts (TIG-1) that were treated with AshLe-WEX or triethylene glycol. Each control group consists of human osteosarcoma cells (U2OS) or normal human fibroblasts (TIG-1) that were not treated with AshLe-WEX or triethylene glycol.

FIG. 12 shows a graph of results of determining telomerase activity in human breast cancer cells (MCF7) that were treated with AshLe-WEX or triethylene glycol. FIG. 12 also shows results for a positive control group of cancer cells having telomerase activity provided with a TRAP assay kit, results for a negative control group of human osteosarcoma cells (U2OS) free of telomerase, and results for a control group of human breast cancer cells (MCF7) that were not treated with AshLe-WEX or triethylene glycol.

FIG. 13 shows photo images of results of investigating the expression levels of Keap1 in human lung cancer cells (A549) that were treated with AshLe-WEX or triethylene glycol. FIG. 13 also shows the expression levels of Keap1 in a control group of human lung cancer cells that were not treated with AshLe-WEX or triethylene glycol.

FIG. 14 shows a photo image and a graph of results of investigating the expression levels of matrix metalloproteases (MMP-9, MMP-3, and MMP-2) in human osteosarcoma cells (U2OS) that were treated with AshLe-WEX or triethylene glycol. FIG. 14 also shows the expression levels of matrix metalloproteases in a control group of human osteosarcoma cells (U2OS) that were not treated with AshLe-WEX or triethylene glycol.

FIG. 15 shows photo images of results of differentiation induction assay of glioblastoma cells (C6 Glioma cells) with the use of AshLe-WEX or triethylene glycol. FIG. 15A shows optical microscopic images of cells subjected to differentiation induction treatment. FIG. 15B shows immunohistochemical images when observing the expression of GFAP (glial cell differentiation marker) in cells subjected to differentiation induction treatment. FIG. 15C shows immunohistochemical images taken at a magnification higher than that in FIG. 15B. A control group consists of cells that were treated with hydrogen peroxide for differentiation induction treatment but not with AshLe-WEX or triethylene glycol.

FIG. 16 shows photo images of results of differentiation induction assay of neuroblastoma cells (IMR32; neuroblastoma cells) using AshLe-WEX or triethylene glycol. FIG. 16A shows optical microscopic images of cells subjected to differentiation induction treatment. FIG. 16B shows immunohistochemical images when observing the expression of the neurofilament protein (NF200) in cells subjected to differentiation induction treatment. In FIGS. 16A and 16B, each control group consists of neuroblastoma cells (IMR32) that were treated with hydrogen peroxide for differentiation induction treatment but not with AshLe-WEX or triethylene glycol. FIG. 16C shows an photo image of Western blotting results of the NF200 expression level in cells subjected to differentiation induction treatment. In FIG. 16C, the leftmost lane corresponds to a control group that was not treated with hydrogen peroxide and AshLe-WEX or triethylene glycol, and the second lane from the left corresponds to a control group that was treated with hydrogen peroxide but not with AshLe-WEX or triethylene glycol.

### DETAILED DESCRIPTION OF THE PREFERRED EMBODIMENTS

The pharmaceutical composition of the present invention is characterized in that it comprises, as an active ingredient, triethylene glycol of formula (I) or a derivative thereof:

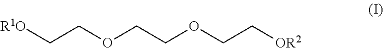

in which R¹ and R² are independently selected from hydrogen, C₁₋₆ alkyl (preferably C₁₋₃ alkyl), C₁₋₆ haloalkyl (preferably C₁₋₃ haloalkyl), or —C(═O)R³, and R³ is selected from C₁₋₆ alkyl (preferably C₁₋₃ alkyl) or C₁₋₆ haloalkyl (preferably C₁₋₃ haloalkyl).

More preferably, R¹ and R² are independently selected from hydrogen, C₁₋₆ alkyl (especially C₁₋₃ alkyl), or C₁₋₆ haloalkyl (especially C₁₋₃ haloalkyl). Further preferably, R¹ and R² are independently selected from hydrogen or C₁₋₆ alkyl, particularly from hydrogen or C₁₋₃ alkyl. In addition, preferably either R¹ or R² is hydrogen.

The present inventors successfully identified that an active ingredient having anticancer activity in water extract of Ashwagandha leaves is triethylene glycol. Based on this finding, the above compound of formula (I) itself or a metabolite generated through in vivo metabolism of the compound is considered to have desired anticancer action.

The term “C₁₋₆ alkyl” used herein refers to a linear or branched saturated hydrocarbon group having 1 to 6 carbon atoms. Examples of C₁₋₆ alkyl include methyl, ethyl, propyl, isopropyl, isobutyl, n-butyl, tert-butyl, isopentyl, and n-pentyl. The term “C₁₋₃ alkyl” used herein refers to methyl, ethyl, propyl, or isopropyl.

The terms “C₁₋₆ haloalkyl” and “C₁₋₃ haloalkyl” refer to C₁₋₆ alkyl and C₁₋₃ alkyl, respectively, in each of which at least one hydrogen has been substituted with a halogen, i.e., fluorine, chlorine, bromine, or iodine.

The term “anticancer activity” used herein refers to an activity of inhibiting cancer growth. More specifically, it means capacity to have cytotoxicity to cancer cells so as to act to, for example, inhibit cancer cell growth and invasion, activate cancer-inhibiting protein p53 or pRB, inhibit telomerase activity, and induce differentiation. The pharmaceutical composition of the present invention can be used alone or in combination with chemotherapy using other anticancer agents or radiation therapy for cancer treatment or prevention. The pharmaceutical composition of the present invention is advantageous in that it acts exclusively on cancer cells and does not substantially affect normal cells.

Further, the present inventors found that triethylene glycol has an action to inhibit the metastasis of cancer cells. The pharmaceutical composition of the present invention can also be used as an agent for inhibiting cancer metastasis for combining with chemotherapy using other anticancer agents or radiation therapy or preventing recurrence of cancer after treatment.

The term “cancer” used herein refers to all forms of cancer, which include: solid cancer such as cancer formed in epithelial tissue (e.g., pancreatic cancer, gastric cancer, large bowel cancer, kidney cancer, liver cancer, bone marrow cancer, adrenal cancer, skin cancer, melanoma, lung cancer, small bowel cancer, prostate cancer, testicular cancer, uterus cancer, breast cancer, or ovarian cancer) or sarcoma that is malignant tumor formed at non-epithelial sites of muscles or bones; and other humoral cancer such as leukemia and malignant lymphoma. The pharmaceutical composition of the present invention is particularly effective for treatment or prevention of solid cancer.

Triethylene glycol has excellent property to retain water molecules, and therefore it is used for a solvent or the like. In addition, triethylene glycol is known as a low-toxic disinfectant having chronic effect against bacteria and viruses in the air, in liquid, or on the surface of an object. Triethylene glycol is commercially available, and thus it can be easily obtained. Also, derivatives of triethylene glycol are commercially available, or those skilled in the art can readily prepare such derivatives by known methods using commercially available reagents. Therefore, the pharmaceutical composition of the present invention can be provided at a relatively low price.

In the pharmaceutical composition of the present invention, the compound of formula (I) as an active ingredient can be formulated in an arbitrary dosage form using pharmaceutically acceptable carriers, if necessary. A variety of dosage forms can be used. Specific examples of dosage forms include tablets, capsules, liquids, powders, fine powders, granules, and injections. The route of administration may be either oral administration or parenteral administration. Examples of parenteral administration include intravenous administration, subcutaneous administration, intramuscular administration, and intraperitoneal administration.

Examples of a pharmaceutically acceptable carrier include excipients, binders, disintegrants, and lubricants for solid formulations. Also, examples thereof include solvents, solubilizing agents, suspending agents, tonicity agents, buffers, and soothing agents for liquid formulations. If necessary, additives to formulations such as preservatives, antioxidants, colorants, sweetening agents, and stabilizers can be used.

An oral solid formulation can be prepared by adding, for example, an excipient, a binder, a disintegrant, a lubricant, a colorant, and/or a flavoring agent, as necessary, to the compound of formula (I) as an active ingredient and formulating the mixture into tablets, granules, capsules, or the like by an ordinary method.

An injection can be prepared by adding, for example, a pH adjuster, a buffer, a stabilizer, a tonicity agent, and/or a local anesthetic to the compound of formula (I) as an active ingredient and formulating the mixture into an injection for intravenous administration, subcutaneous administration, intramuscular administration, or intraperitoneal administration by an ordinary method.

The present invention also relates to a method for treating or preventing cancer which encompasses administering an effective amount of triethylene glycol according to formula (I) or a derivative thereof to mammals, especially humans, in need of cancer treatment. The term “effective amount” refers to, for example, the amount of an active ingredient at which a biological or medical response of a tissue, system, animal, or human is induced to a desirable extent for a researcher or a clinical physician. The effective amount of the compound of formula (I) as an active ingredient according to the present invention may vary depending on the age, body weight, and severity of the subject of administration, formulation properties, route of administration, etc. The effective amount of the compound of formula (I) for treatment of mammals, especially humans, is, for example, 50 to 500 mg/kg/day, preferably 50 to 250 mg/kg/day, and particularly preferably 100 to 250 mg/kg/day for oral administration, and 50 to 500 mg/kg/day, preferably 50 to 250 mg/kg/day, and particularly preferably 50 to 200 mg/kg/day for parenteral administration. It is preferable to administer the effective amount of the compound once a day or in two or three times a day by dividing the amount. The unit dose of an oral or parenteral preparation contains preferably 10 to 500 mg, particularly 50 to 250 mg of the compound according to formula (I).

### EXAMPLES

The present invention is described in more detail with reference to the Examples below. However, the present invention is not limited to the Examples.

### 1. Preparation of a Water Extract of Ashwagandha Leaves (AshLe-WEX)

10 g of a powder of dried Ashwagandha (Withania somnifera) leaves (originating in India, purchased from iGENE) was added to 100 mL of water to prepare a 10% suspension. The suspension was placed in an incubator at 45° C. and slowly shaken overnight for extraction treatment. The suspension subjected to extraction treatment was centrifuged at 10,000 rpm for 20 minutes. The supernatant thereof was filtered via a 0.45 μm filter to obtain a water extract of Ashwagandha leaves (AshLe-WEX).

### 2. Cytotoxicity Assay of AshLe-WEX on Cancer Cells

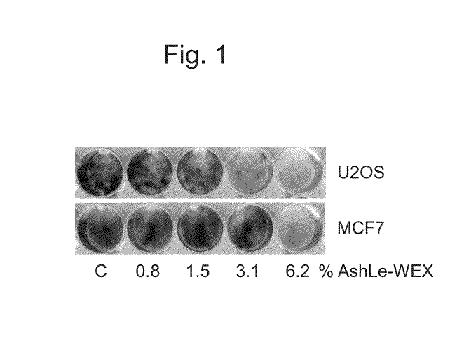

AshLe-WEX was added to mediums culturing human osteosarcoma cells (U2OS, obtained from the American Type Culture Collection) or human breast cancer cells (MCF7, obtained from the JCRB Cell Bank) such that a final concentration thereof is within a range of 0.8% to 6.2%. After the addition of AshLe-WEX, culture was carried out at 37° C. for 48 hours, followed by staining of cells with crystal violet. A group of cells cultured in the above manner in a medium without adding AshLe-WEX was designated as a control group. As shown in FIG. 1, AshLe-WEX exhibited cytotoxicity to both cancer cells tested above.

### 3. Inactivation of Protein Components of AshLe-WEX

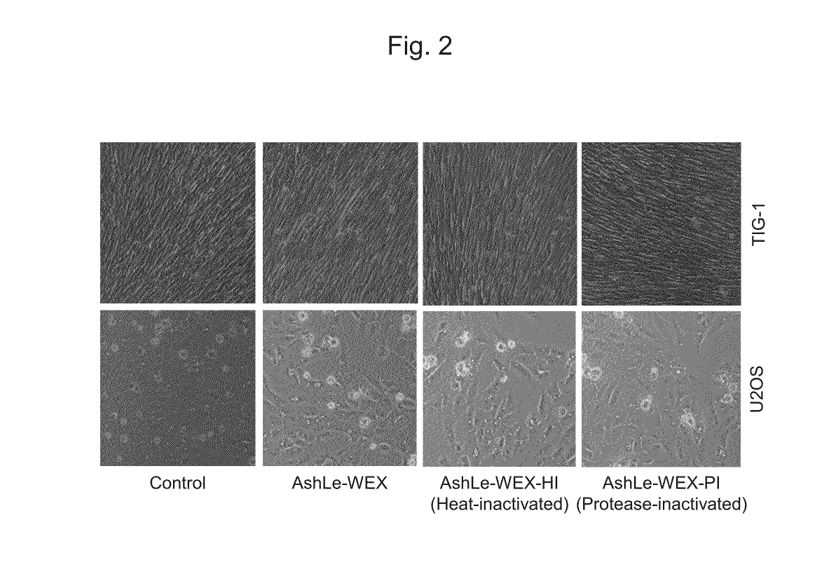

In order to identify an anti-cancer active component of AshLe-WEX, samples of AshLe-WEX in which protein components had been inactivated by heat denaturation or proteinase degradation (AshLe-WEX-HI and AshLe-WEX-PI) were prepared. AshLe-WEX and the samples whose protein components were inactivated were used for testing cytotoxicity to human osteosarcoma cells (U2OS) and normal human fibroblasts (TIG-1, obtained from the JCRB Cell Bank). FIG. 2 shows the results. According to the test, it was found that the cytotoxicity of AshLe-WEX is independent from its protein components.

### 4. Fractionation of AshLe-WEX

AshLe-WEX obtained in item 1 above was fractionated by reversed-phase HPLC using C18 column (TSKgel ODS-100Z, Tosoh Corporation). The flow rate was 1 mL/minute, the column temperature was 40° C., and the detection wavelength was 220 nm. Gradient elution was carried out using water as solution A and ethanol as solution B under the following conditions.

Until 5 minutes: Solution A, 100% (constant)

Until 20 minutes: Gradient, up to 0.75% of solution B

Until 25 minutes: Gradient, from 0.75% to 50% of solution B

Until 30 minutes: Solution B, 50% (constant)

Until 32 minutes: Gradient, from 50% to 0% of solution B

Until 37 minutes: Solution A, 100% (constant)

As a result of gradient elution, AshLe-WEX was successfully fractionated into four fractions (AshLe-WEX-F1 to F4).

### 5. Cytotoxicity Assay of AshLe-WEX and the Fractions Thereof

Human osteosarcoma cells (U2OS) were cultured in a humidified incubator (37° C., 5% CO₂) using a medium in which Dulbecco's modified Eagle's medium (DMEM, Invitrogen) is added with 10% bovine fetal serum. The cells were cultured to become 40% to 60% confluent and treated with AshLe-WEX (final concentration: 1%; 200 μg/mL) and the 1st and 2nd fractions thereof (AshLe-WEX-F1 and F2), respectively. The cells were treated over about 48 hours during culture at 37° C.

AshLe-WEX and the fractions thereof were evaluated in terms of cytotoxicity by assay using MTT. After the above treatment, MTT (0.5 mg/mL) was added to each cell culture medium, followed by incubation for 4 hours. Next, the medium containing MTT was removed and 100 μL of DMSO was added to each well to completely dissolve formazan crystals. Absorbance was detected at 550 nm using a spectrophotometer (Wallac ARVO SX). As a result of cytotoxicity assay, the 2nd fraction (AshLe-WEX-F2) was found to contain an anti-cancer active component.

### 6. Identification of the Anti-Cancer Active Component

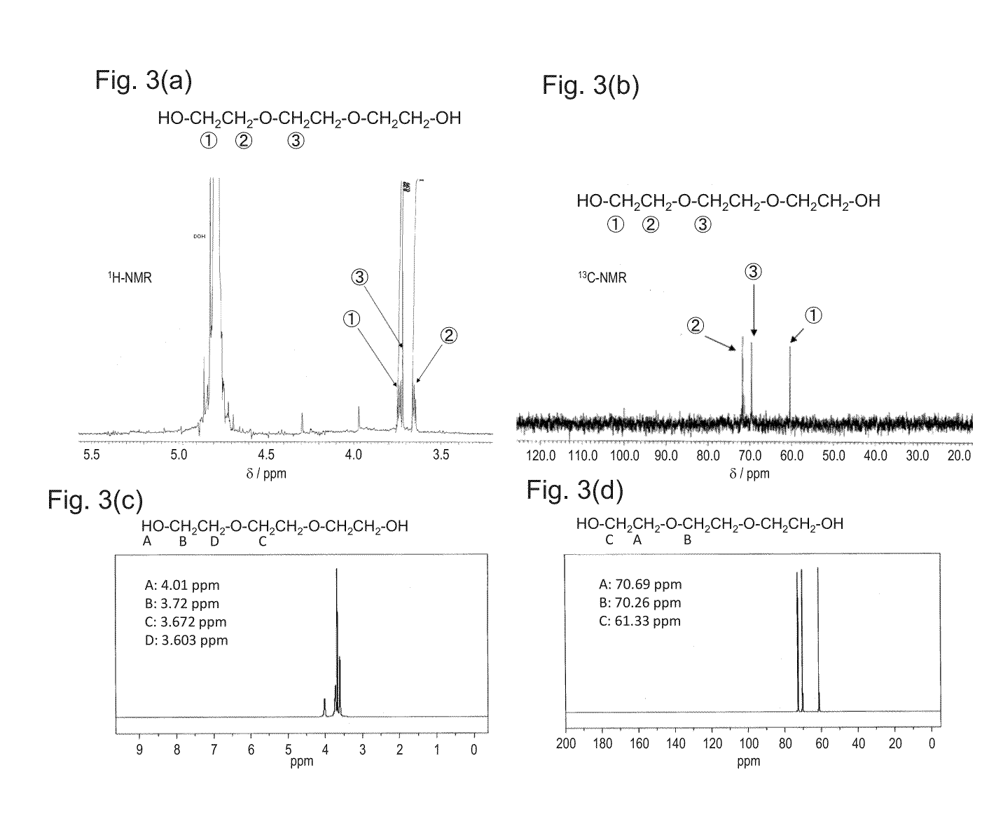

AshLe-WEX-F2 was subjected to heat denaturation of proteins, dried, and dissolved in deuterated water and NMR analysis (¹H-NMR and ¹³C-NMR) was carried out. The obtained spectra are shown in FIGS. 3((a) and (b)). The main component of AshLe-WEX-F2 was confirmed to be triethylene glycol (TEG) by comparing the spectra with known spectral data (FIGS. 3(c) and (d)).

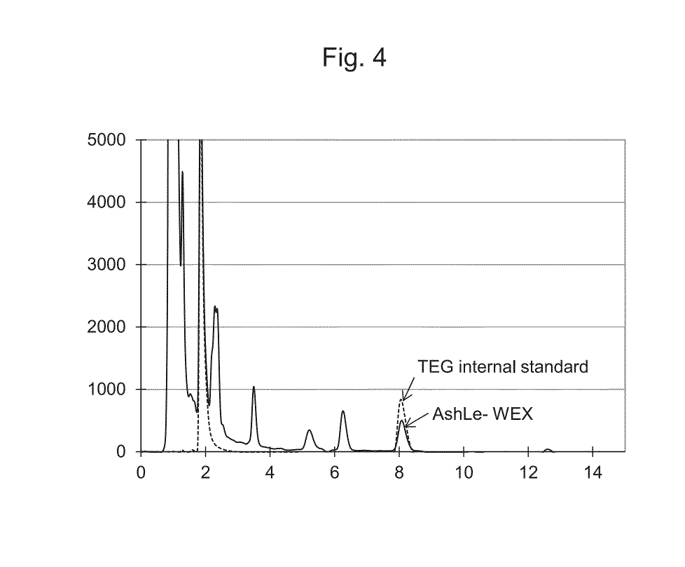

In order to confirm the presence of triethylene glycol in AshLe-WEX, HPLC analysis was conducted at 40° C. using water as a mobile phase (injection volume: 10 μL; flow rate: 2 mL/minute) and LUNA C18(2) column (length: 150 mm; inner diameter: 4.6 mm; particle size: 5 μm; Phenomenex) and a refractive index detector RID-10A (Shimadzu Corporation). Commercially available purified triethylene glycol was used as a standard substance. FIG. 4 shows the obtained chart. The analysis results confirmed that AshLe-WEX contains triethylene glycol.

### 7. Cytotoxicity Assay of Triethylene Glycol (TEG)

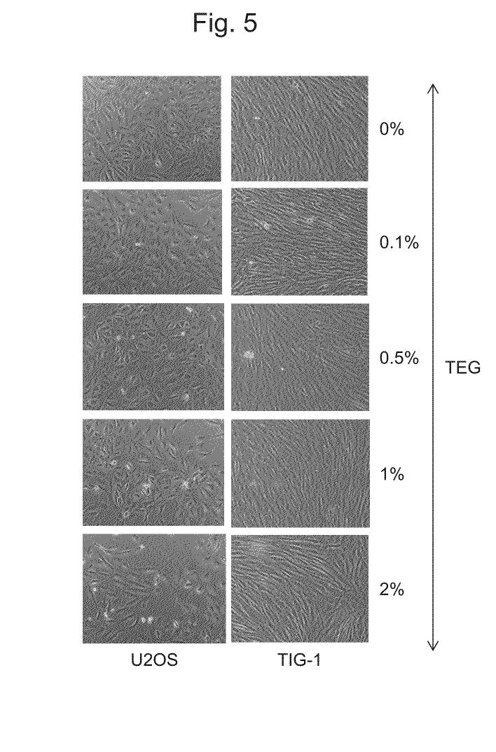

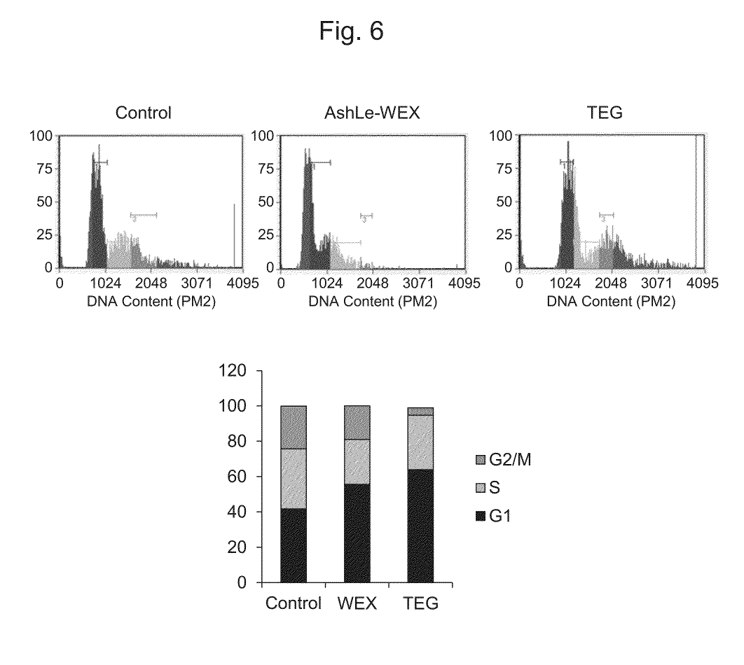

Cytotoxicity of triethylene glycol to human osteosarcoma cells (U2OS) and normal human fibroblasts (TIG-1) was tested in the manner described in item 5 above. FIG. 5 shows the results. It was found that triethylene glycol inhibits cancer cell growth at a concentration of 0.5% or more, while it has substantially no toxicity to normal human fibroblasts. Further, cell cycle analysis was conducted using human osteosarcoma cells (U2OS). As a result, both AshLe-WEX and triethylene glycol were found to cause G1 arrest (FIG. 6). That is, AshLe-WEX and triethylene glycol were found to have an effect of arresting the cell cycle of cancer cells specifically at the G1 phase.

### 8. In Vivo Antitumor Assay

In vivo antitumor assay of AshLe-WEX and triethylene glycol was conducted using subcutaneous xenograft and tail vein metastasis model mice produced with the use of HT1080 cells from an invasive tumor with high lung metastasis (human fibrosarcoma cells, obtained from the JCRB Cell Bank).

HT1080 cells (6×10⁶ cells in 0.2 mL of growth medium) were subcutaneously injected into Balb/c nude mice (4-week-old female mice purchased from CLEA Japan, Inc.) at two sites per mouse, and an equivalent amount of the same was injected into the tail vein of each mouse. 2% carboxymethyl cellulose was administered with feed to a control group. A mixture containing AshLe-WEX (100-250 mg/kg body weight/administration) and 2% carboxymethyl cellulose was administered with feed to an AshLe-WEX group. A TEG group was given a 5% triethylene glycol aqueous solution via oral administration (at a dose per administration of 250 μL of a preparation of 5% triethylene glycol mixed with 2% carboxymethyl cellulose) or intraperitoneal injection (at a dose per administration of 100 μL of 5% triethylene glycol). This administration treatment was started on day 8 after injection of HT1080 cells and repeated 12 times in total at intervals of two days. Tumorigenesis was observed for one month to calculate the subcutaneous tumor volume. For metastasis assay, each mouse was euthanized by cervical dislocation 5 weeks after tail vein injection, the lung was fixed with 4% formaldehyde, and the number of tumor colonies was counted. This assay was repeated twice using three mice for each group.

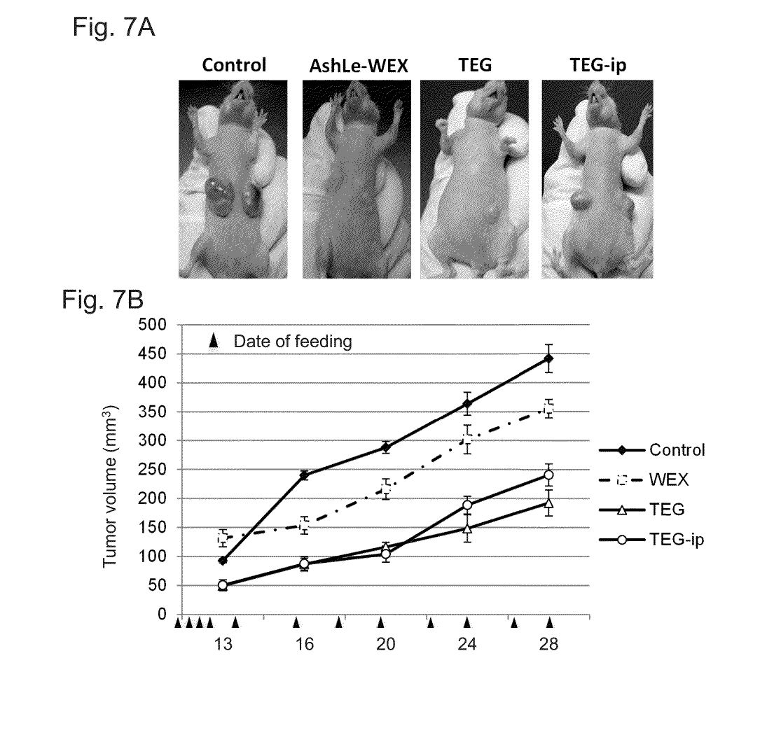

FIG. 7 shows the results. An effect of tumor growth inhibition was confirmed in the group of mice orally administrated with triethylene glycol by feeding (TEG group). A similar effect of inhibiting tumor growth was observed in the group subjected to intraperitoneal injection (TEG-ip group). Namely, both oral administration and intraperitoneal injection of triethylene glycol were found to have an effect of remarkably suppressing an increase in tumor volume due to tumor growth.

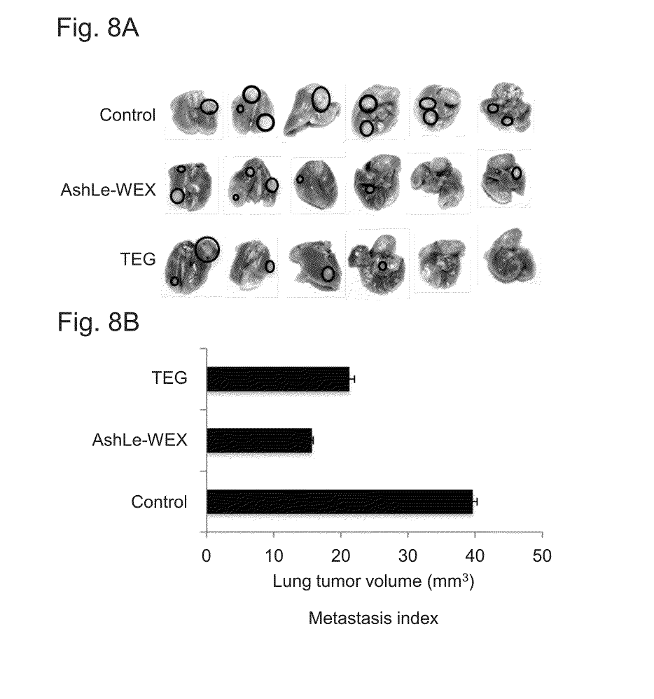

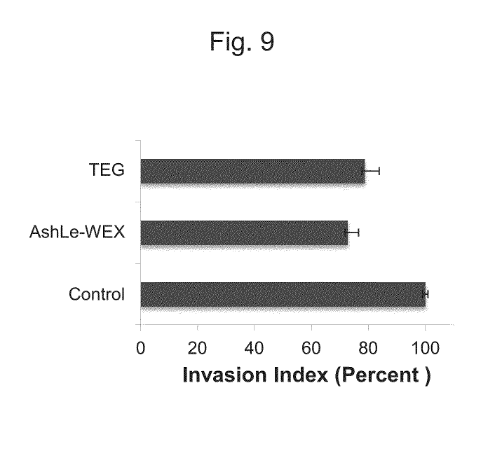

In addition, triethylene glycol exhibited a powerful antimetastatic activity. FIG. 8 shows results of in vivo tumor metastasis assay. FIG. 8A shows photo image data of the lungs excised from mice after in vivo tumor metastasis assay, in which each circled area indicates a tumor formed through metastasis. In FIG. 8A, the upper photos are of the 2% carboxymethyl cellulose administration group (control group), the middle photos are of the AshLe-WEX administration group, and the bottom photos are of the triethylene glycol administration group. FIG. 8B shows a graph of the mean lung tumor volumes for each group after in vivo antitumor assay. Bulky tumors were found in the lungs of all mice of the control group. Meanwhile, the number of lung tumors in mice treated with AshLe-WEX or triethylene glycol was lower than that of the control group, and the tumor volumes of the treated mice were significantly low. In addition, as a result of in vitro Matrigel invasion assay of HT1080 cells treated with AshLe-WEX or TEG, invasion was found to be reduced (FIG. 9). These results suggested that triethylene glycol is the major antitumor factor of AshLe-WEX.

### 9. Anti-Cancer Activity Mechanism of Triethylene Glycol

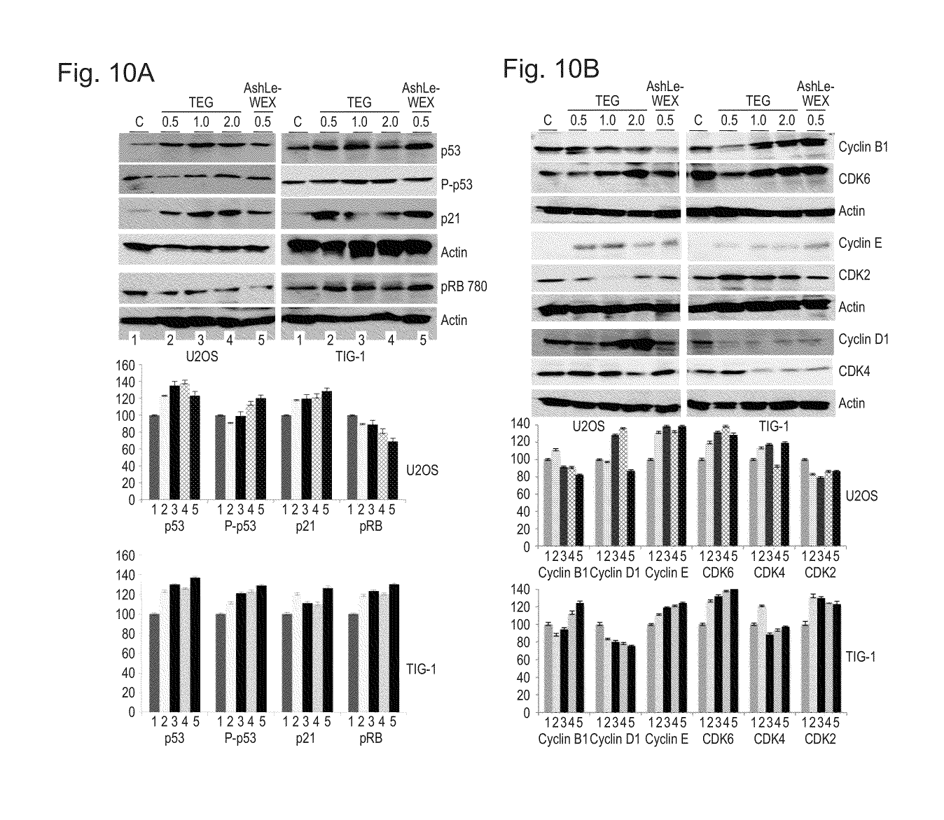

In order to investigate the cytotoxicity action mechanism of triethylene glycol, human osteosarcoma cells (U2OS) and normal human fibroblasts (TIG-1) were each treated with AshLe-WEX or triethylene glycol (TEG), and the expression of cancer-inhibiting proteins p53 and pRB was examined by SDS-PAGE and Western blotting. Treatment with AshLe-WEX or triethylene glycol (TEG) was conducted by adding AshLe-WEX (final concentration: 0.5%) or triethylene glycol (final concentration: 0.5%, 1.0%, or 2.0%) to a medium for culturing cells and culturing for 48 hours. FIG. 10A shows the results. Numbers 1-5 in the bar charts of the expression level in the lower half of FIG. 10A correspond to numbers 1-5 of the lanes in the photo images in the upper half of FIG. 10A. The expression level of p53 in U2OS cells treated with AshLe-WEX or triethylene glycol was found to have increased as compared with that of the control group. In addition, the expression levels of p53 and p21 in normal cells treated with AshLe-WEX or triethylene glycol were found to have increased as compared with those of the control group. Further, as a result of a test using an anti-phosphoserine-specific antibody, it was revealed that the phosphorylated-p53 protein increased in both cancer cells and normal cells. It was found based on the proportion of phosphorylated-p53/p53 that phosphorylation rate increased in cells treated with AshLe-WEX or triethylene glycol by 30% to 40%, as compared with the control group. This suggests that AshLe-WEX and triethylene glycol can activate p53 in both cancer cells and normal cells. Meanwhile, regarding pRB, it was found that phosphorylated-pRB decreased in cancer cells treated with AshLe-WEX or triethylene glycol while it increased in normal cells treated with the same, as compared with the control group. The proportion of phosphorylated-pRB/RB decreased from about 0.5 in control cells to about 0.3 in cells treated with triethylene glycol (about 20% decrease) while it increased by about 20% in normal cells in contrast.

In parallel with the above, the expression levels of cyclin-B1, -D1, and -E1 and CDK-2, -4, and -6 were examined. FIG. 10B shows the results. The expression level of cyclin-B1 in cancer cells treated with AshLe-WEX or triethylene glycol was found to have decreased, while on the other hand, the expression level of cyclin-B1 in normal cells was found to have increased as compared with that in the control group. On the other hand, the tendency regarding the expression levels for cyclin-D1 was the opposite of that for cyclin-B1. In the case of treatment with triethylene glycol, the expression level of cyclin-D1 increased in cancer cells but decreased in normal cells. The expression level of cyclin-E1 increased in both cancer cells and normal cells. The expression level of CDK-4 decreased in normal cells.

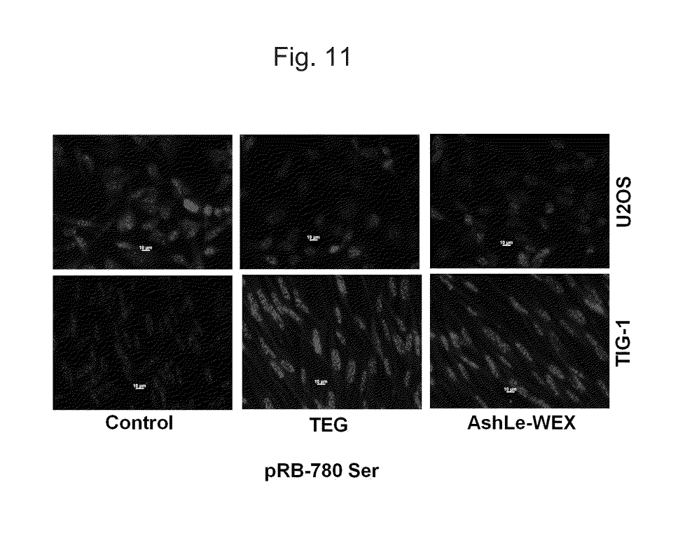

Immunohistochemistry was conducted for visualizing the degree of phosphorylation of p53 and pRB in human osteosarcoma cells (U2OS) and normal human fibroblasts (TIG-1) treated with AshLe-WEX or triethylene glycol. Treatment with AshLe-WEX or triethylene glycol was conducted by adding AshLe-WEX or triethylene glycol to a medium such that a final concentration becomes 0.5% and culturing the cells in each medium for 48 hours. The cells were stained with an anti-p53 antibody (DO-1) and an anti-pRB antibody (S780). Immunostaining was visualized using an Alexa-488- or Alexa-594-labeled secondary antibody. Regarding pRB, which is a downstream effector of a cyclin-CDK complex, the degree of phosphorylation decreased in cancer cells treated with AshLe-WEX or triethylene glycol but increased in normal cells treated with AshLe-WEX or triethylene glycol (FIG. 11).

### 10. Determination of Telomerase Activity

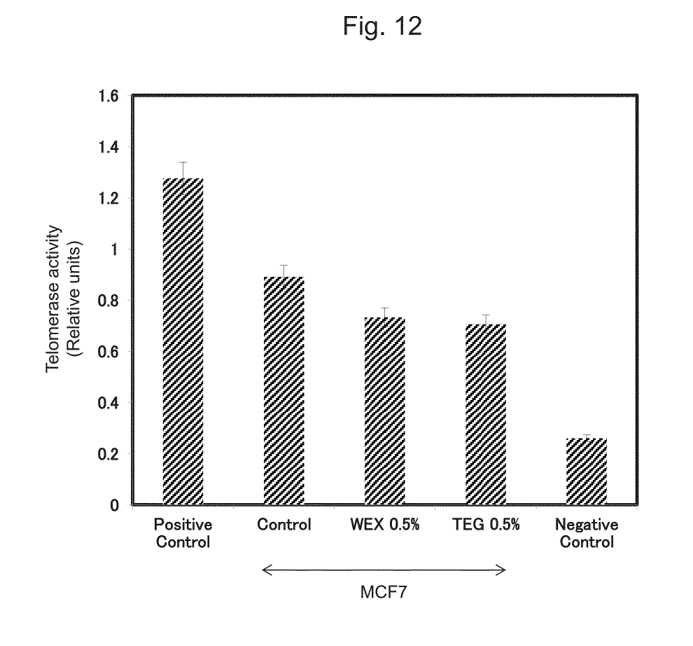

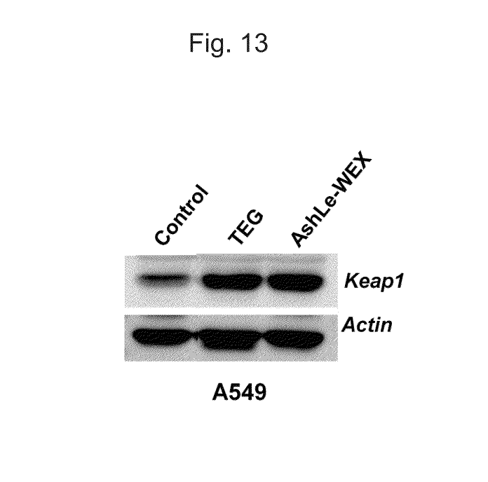

Effects of triethylene glycol upon telomerase activity, that is an established feature of cancer cells, were examined using a TRAP assay kit (TeloTAGGG telomerase PCR ELISA PLUS; purchased from Roche Applied Science; Cat #12 013 789 001). The cells used herein were human breast cancer cells (MCF7). Human breast cancer cells were cultured in a medium containing AshLe-WEX or triethylene glycol at a final concentration of 0.5% for 48 hours and then used for determination of telomerase activity. FIG. 12 shows the examination results. Triethylene glycol and AshLe-WEX were found to inhibit telomerase activity at a rate of 20% to 40%. In addition, Western blotting assay results shown in FIG. 13 revealed that triethylene glycol and AshLe-WEX cause increase in the expression level of Keap1, which activates a transcription factor (NRF2) in an oxidation stress depending manner.

### 11. Antimetastatic Activity Mechanism of Triethylene Glycol

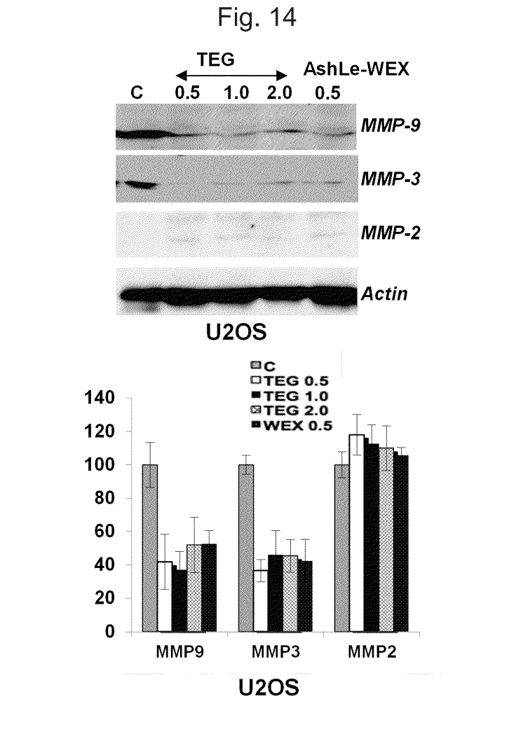

In order to examine the antimetastatic activity mechanism of AshLe-WEX and triethylene glycol, the expression levels of matrix metalloproteases (MMP-9, MMP-3, and MMP-2) were examined. Human lung cancer cells (A549) were used for examination. Human lung cancer cells (A549) were cultured in a medium containing AshLe-WEX (final concentration: 0.5%) or triethylene glycol (final concentration: 0.5%, 1.0%, or 2.0%) for 48 hours and then used for determination of the expression levels of matrix metalloproteases. As shown in FIG. 14, the expression levels of MMP-3 and MMP-9 in cancer cells treated with AshLe-WEX and TEG remarkably decreased, suggesting antimetastatic activity observed in the aforementioned in vitro and in vivo assays. This effect was not observed for MMP-2.

### 12. Differentiation Induction Activity of Triethylene Glycol

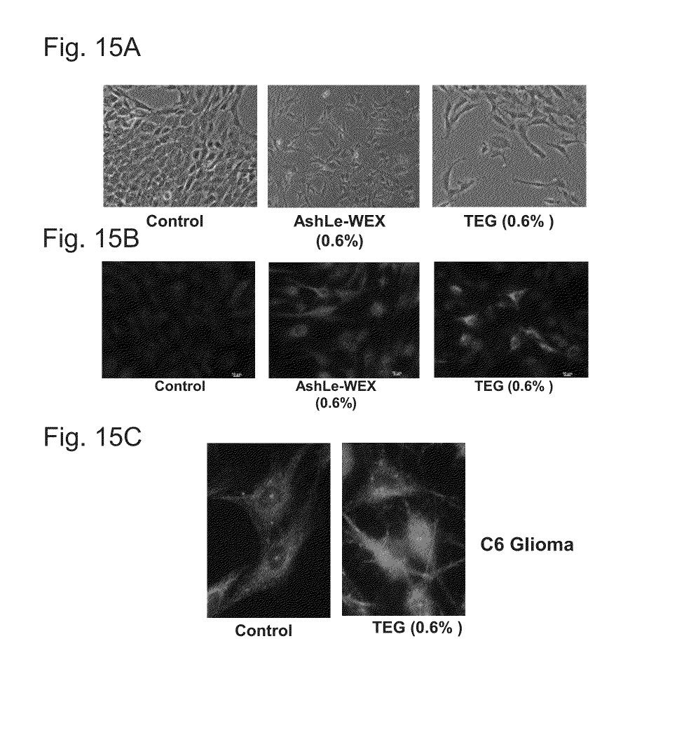

Differentiation induction of glioblastoma cells and neuroblastoma cells by triethylene glycol was examined. Differentiation of glioblastoma cells was induced by treating the cells with 100 μmol of hydrogen peroxide for 2 to 3 hours and then culturing the cells in a medium containing AshLe-WEX or triethylene glycol (final concentration: 0.6%) for 48 hours. Cells of a control group were treated with hydrogen peroxide in the above manner and then cultured in a medium containing neither AshLe-WEX nor triethylene glycol. FIG. 15 shows differentiation induction assay results for glioblastoma cells. As shown in FIG. 15A, glioblastoma cells treated with AshLe-WEX or triethylene glycol were in an astrocyte-like form, indicating differentiation induction. Further, induction of GFAP (glial cell differentiation marker protein) was upregulated in cells treated with AshLe-WEX or triethylene glycol (FIG. 15B). An astrocyte-like form was found in a highly magnified photo image of differentiated cells, which indicates a high expression level of GFAP (FIG. 15C).

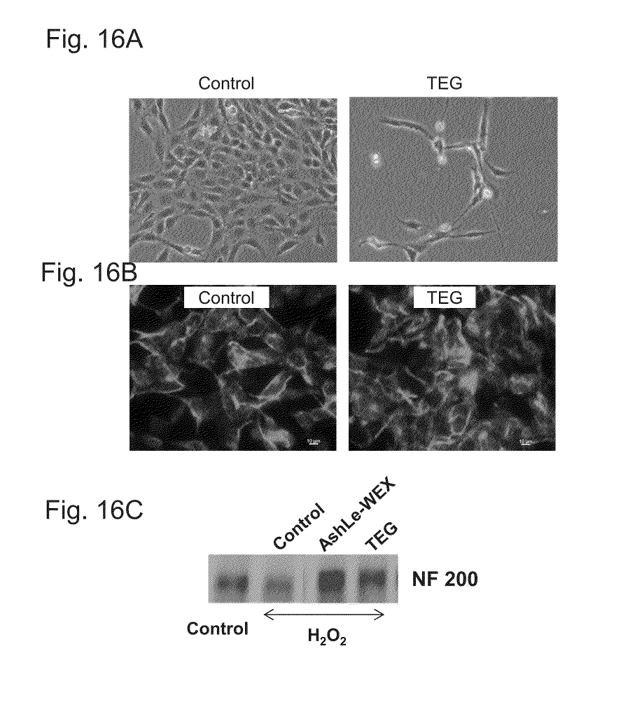

Next, effects on IMR32 neuroblastoma cells treated with AshLe-WEX or triethylene glycol were examined. Differentiation of neuroblastoma cells was induced by treating the neuroblastoma cells with 100 μmol of hydrogen peroxide for 2 to 3 hours and then culturing the cells in a medium containing AshLe-WEX or triethylene glycol (final concentration: 0.5%). As control groups, a group of cells that had been treated with hydrogen peroxide in the above manner and then cultured in a medium containing neither AshLe-WEX nor triethylene glycol and a group of cells that no hydrogen peroxide treatment was conducted and cultured in a medium containing neither AshLe-WEX nor triethylene glycol without hydrogen peroxide treatment were prepared. FIG. 16 shows the results. IMR32 treated with AshLe-WEX or triethylene glycol was in a neuron-like form, indicating an increase of a neurofilament protein (NF200) (FIGS. 16A, 16B, and 16C).

All publications, patents, and patent applications cited herein are incorporated herein by reference in their entirety.

What is claimed is:

1. A pharmaceutical composition for treating or preventing a cancer, which comprises, as an active ingredient, a triethylene glycol of formula (I) or a derivative thereof: 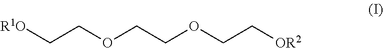
wherein
R¹ and R² are independently selected from hydrogen, C₁₋₆ alkyl, C₁₋₆ haloalkyl, or C(═O)R³, and
R³ is selected from C₁₋₆ alkyl or C₁₋₆ haloalkyl.

2. The pharmaceutical composition according to claim 1, wherein the cancer is a solid cancer.

3. An agent for inhibiting cancer metastasis, which comprises, as an active ingredient, a triethylene glycol of formula (I) or a derivative thereof: 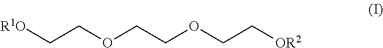
wherein
R¹ and R² are independently selected from hydrogen, C₁₋₆ alkyl, C₁₋₆ haloalkyl, or C(═O)R³, and
R³ is selected from C₁₋₆ alkyl or C₁₋₆ haloalkyl.

4. A method for treating or preventing cancer, which comprises administering to a subject in need thereof an effective amount of a triethylene glycol of formula (I) or a derivative thereof: 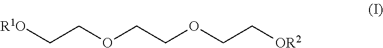
wherein
R¹ and R² are independently selected from hydrogen, C₁₋₆ alkyl, C₁₋₆ haloalkyl, or —C(═O)R³, and
R³ is selected from C₁₋₆ alkyl or C₁₋₆ haloalkyl.
